(19) United States
(12) Patent Application Publication
(10) Pub. No.: US 2021/0137823 A1
(43) Pub. Date: May 13, 2021

(54) SKIN WHITENING PROGRAM AND APPLICATION THEREOF

(71) Applicant: INFINITUS (CHINA) COMPANY LTD., Jiang Men, Guangdong (CN)

(72) Inventors: Jian TANG, Jiang Men, Guangdong (CN); Yafei CHEN, Jiang Men, Guangdong (CN); Yanshan LIANG, Jiang Men, Guangdong (CN)

(73) Assignee: INFINITUS (CHINA) COMPANY LTD., Jiang Men, Guangdong (CN)

(21) Appl. No.: 16/493,220

(22) PCT Filed: May 16, 2017
(86) PCT No.: PCT/CN2017/084493

§ 371 Date: Sep. 11, 2019

(30) Foreign Application Priority Data
Apr. 26, 2017 (CN) 201710283287.7

(51) Int. Cl. A61K 8/9789; A61K 8/9728; A61K 8/98; A61Q 19/02; A61K 8/9771; A61K 8/02; A61H 7/00; A61N 1/32; A61N 5/06; A23L 33/105; A23L 33/00

(52) U.S. Cl. CPC A61K 8/9789; A23V 2002/00; A61K 8/987; A61Q 19/02; A61K 8/9771; A61K 8/0212; A61H 7/003; A61N 1/328; A61N 5/0616; A23L 33/105; A23L 33/40; A61N 2005/0663; A61N 2005/0651; A61H 2201/105; A61H 2201/10; A61K 8/9728

(57) ABSTRACT

A skin whitening program, comprising an external application step, a treatment step and an oral administration step. A skin care product for external application is selected from a whitening, brightening or yellow-removing skin care product, the effective ingredient in the skin care product being selected from a tricholoma matsutake, carthamus tinctorius or rhodiola rosea extract, a tricholoma matsutake, glycyrrhiza uralensis or pearl extract, or a panax notoginseng, salvia miltiorrhiza or radix astragali extract. A beautifying device for treatment is selected from an iontophoresis, microcurrent or LED green light beautifying device. A beautifying food for oral administration is selected from an antioxidant, blood and qi invigorating or anti-glycation food, the effective ingredient in the food being selected from a schisandra chinensis, panax quinquefolius or ophiopogon japonicus extract, a salvia miltiorrhiza, crataegus pinnatifida or ginkgo biloba extract, or an olive, osmanthus fragrans or scutellaria baicalensis extract.

This application claims the priority of Chinese Patent Application No. 201710283287.7, filed with China National Intellectual Property Administration on Apr. 26, 2017, and titled with “SKIN WHITENING PROGRAM AND APPLICATION THEREOF”, and the disclosure of which is hereby incorporated by reference.

### FIELD

The present disclosure relates to the field of skin care, specifically to a method for delaying aging of skin and the use thereof.

### BACKGROUND

Loving beauty is a common problem for human beings, especially for women. As the economy increases year by year, more and more women pay special attention to their appearance. Among them, skin care, as an important part of beauty, accounts for more than half of the women's beauty consumption. Skin care has become a science in modern times. People troubled by skin problems no longer aimlessly go to stores to buy a variety of cosmetics to try, but are prescribed by a dermatologist who provides targeted skin care advice. With the increasing pursuit of health and the rapid development of dermatology, medical skin care products with higher safety and effectiveness have become an irresistible trend. With more women beginning to pay attention to the quality and safety of skin care products, the professionals have deeper research and understanding of the structure and physiological functions of skin and its appendages, the research on cosmetics is more specialized, and the performance is more targeted, reliable and competitive.

However, for some problem skin, the skin problem looks like a localized change, but is essentially an external manifestation of the overall imbalance. Skin care cannot be effective with a single solution or even an extreme solution. In theory, there is no single treatment that is able to achieve the overall beauty from the local to the whole body. Beauty must be holistic, and the treatment of beauty must also be considered as a whole. At present, there are many means for becoming beautiful, including skin care products, medicines, oral beauty foods and various aesthetic medicine technologies. From the perspective of technology, each mean has its best indication. However, traditional therapeutic solutions often attempt to treat skin problems of different levels of development or different types with a single technique, often ignoring the synergy of other related technologies and the role of multidisciplinary treatment. As a result, the overuse of a certain technology results in an unsatisfactory effect. In the face of a variety of beautiful options to choose from, a combination of a variety of technical means helps improving beauty. Therefore, how to classify and combine the available beautiful technologies and programs, learn from each other's strengths, and realize the overall beauty is an urgent requirement for the comprehensive application of multidisciplinary and cross-category.

Thus, it has become a problem urgently to be solved by one of ordinary skill in the art that researching and developing a skin whitening program and use thereof to solve the technical defects that various skin care methods cannot be reasonably combined to achieve an ideal skin care effect in the conventional art.

### SUMMARY

In view of this, the present disclosure provides a skin whitening program and use thereof, which are used for solving the technical defects that various skin care methods cannot be reasonably combined to achieve an ideal skin care effect in the conventional art.

A skin whitening program, comprising

step 1, external application: applying a toner to face 1-3 times each day after cleaning the face, and the time interval between applications is 8-24 h;

step 2, introducing treatment: while performing the external application, using an importer to massage the face, after introducing, successively using an essence milk and a cream;

step 3, oral administration: after the above steps, orally administering a beauty food for 1-3 times each day, and the time interval between oral administrations is 8-24 h;

the skin care product is selected from the group consisting of a skin whitening product, a skin brightening product and a skin yellow-removing product, and a mixture thereof;

the beauty device is selected from the group consisting of an ion beauty importer, a microcurrent beauty device, an LED green light beauty device, and a mixture thereof;

the beauty food is selected from the group consisting of an antioxidant food, a qi-blood promoting food, an anti-saccharification food, and a mixture thereof.

Preferably, the effective ingredient of the skin whitening product comprises one or more of TRICHOLOMA MATSUTAKE extract, CARTHAMUS TINCTORIUS (SAFFLOWER) FLOWER extract, and RHODIOLA ROSEA extract, the content of the effective ingredient in the skin whitening product is 10-30% by mass;

the effective ingredient of the skin brightening product comprises one or more of TRICHOLOMA MATSUTAKE extract, GLYCYRRHIZA URALENSIS extract and PEARL extract, the content of the effective ingredient in the skin moisturizing product is 10-30% by mass;

the effective ingredient of the skin yellow-removing product comprises one or more of PANAX NOTOGINSENG extract, SALVIA MILTIORRHIZA ROOT extract and ASTRAGALUS MEMBRANACEUS extract, the content of the effective ingredient in the skin recovering product is 10-30% by mass.

Preferably, the effective ingredient of the antioxidant food comprises one or more of SCHISANDRAE CHINENSIS extract, PANAX QUINQUEFOLIUS extract and BAKUMONDOU EKISU extract, the content of the effective ingredient in the antioxidant food is 10-30% by mass;

the effective ingredient of the qi-blood promoting food comprises one or more of SALVIA MILTIORRHIZA ROOT extract, CRATAEGI FRUCTUS extract and GINKGO BILOBA extract, the content of the effective ingredient in the collagen-supplementing food is 10-30% by mass; and

the effective ingredients of the anti-saccharification food comprises one or more of CANARIUM ALBUM extract, OSMANTHUS FRAGRANS extract and SCUTELLARIAE RADIX extract, the content of the effective ingredient in the qi-blood promoting food is 10-30% by mass.

Preferably, in the external application step, the program of applying toner is: soaking mask paper and/or cotton pad in the toner for 3-10 min, and applying the soaked mask paper and/or cotton pad to the face for 10-30 min.

Preferably, in the external application step, an isolation layer is configured on the exterior of the paper mask and/or cotton pad when applying the soaked paper mask and/or cotton pad to the face.

Preferably, the isolation layer is selected from the group consisting of a plastic film, a fiber film and a pearlescent film.

Preferably, in the external application step, preheating the toner before applying to the face;

the temperature for preheating is 30-40° C.

Preferably, in the external application step, massaging the face by pressing and/or gently rubbing while applying to the face.

Preferably, the duration of single introducing treatment is 3-20 min.

Preferably, the amount of the toner for single use is 2-5 mL.

Preferably, in the oral administration step, orally taking the health care product with 100-300 mL water.

The present disclosure also provides a use of the skin whitening program according to any one of above in the field of skin care, skin recovery and skin treatment.

In view of above, the present disclosure provides a skin whitening program, comprising: step 1, external application: applying a toner to face 1-3 times each day after cleaning the face, and the time interval between applications is 8-24 h; step 2, introducing treatment: while performing the external application, using an importer to massage the face, after introducing, successively using an essence milk and a cream; step 3, oral administration: after the above steps, orally administering a beauty food for 1-3 times each day, and the time interval between oral administrations is 8-24 h; the skin care product is selected from the group consisting of a skin whitening product, a skin brightening product and a skin yellow-removing product, and a mixture thereof; the beauty device is selected from the group consisting of an ion beauty importer, a microcurrent beauty device, an LED green light beauty device, and a mixture thereof; and the beauty food is selected from the group consisting of an antioxidant food, a qi-blood promoting food and an anti-saccharification food, and a mixture thereof. The present disclosure also provides a use of the above-mentioned skin whitening program in the field of skin care, skin recovery and skin treatment. It can be concluded from the volunteer recruitment test that after treating skin with the program provided by the present disclosure, skin condition of the volunteers improved obviously, and the area of wrinkle on the surface of skin was reduced. The skin whitening program and use thereof provided by the present disclosure solves the technical defects that various skin care methods cannot be reasonably combined to achieve an ideal skin care effect in the conventional art.

### DETAILED DESCRIPTION

The examples of the present disclosure provides a skin whitening program and use thereof, which are used for solving the technical defects that various skin care methods cannot be reasonably combined to achieve an ideal skin care effect in the conventional art.

The technical solutions in the examples of the present disclosure will be clearly and completely described below. It is apparent that the described examples are only a part of the examples of the present disclosure instead of all of the examples. All other embodiments obtained by one of ordinary skill in the art based on the examples of the present disclosure without creative efforts are within the scope of protection of the present disclosure.

In order to illustrate the present disclosure in more details, the skin whitening program and use thereof provided by the present disclosure will be specifically described with examples hereinafter.

### Example

Female volunteers aged 25-49 were recruited. 60 female volunteers who have similar skin types, similar requirements for skin whitening without serious allergy were selected via questionnaire, instrument detection and examination by technical staffs. The volunteers were randomly divided into 6 groups, and there were 10 volunteers in each group.

Double-blind experiment was carried out on each group of volunteers. In blank control group, the skin care product was replaced by a basic moisturizing set (wherein the toner was replaced by distilled water), and the beauty food was replaced by starch pill. The treatment programs of test groups are shown in Table 1.

TABLE 1
Treatment Programs

| | Grope 1 | | Grope 2 | | Grope 3 | | Grope 4 | | Grope 5 | | Grope 6 | |
|---|---|---|---|---|---|---|---|---|---|---|---|---|
| Skin care product | Skin | | Skin | | Skin | | Skin | | Basic | | Basic | |
| | whitening | | brightening | | yellow- | | whitening | | moisturizing | | moisturizing | |
| | product | | product | | removing | | product | | skin care | | skin care | |
| | | | | | product | | | | product | | product | |
| Toner | Skin | | Skin | | Skin | | Skin | | Distilled | | Distilled | |
| | whitening | | brightening | | yellow- | | whitening | | water | | water | |
| | toner | | toner | | removing | | toner | | | | | |
| | | | | | toner | | | | | | | |
| Times of | 1 | time | 2 | times | 3 | times | 1 | time | 2 | times | 3 | times |
| Applying to Face | | | | | | | | | | | | |
| Time Interval | 24 | h | 12 | h | 8 | h | 24 | h | 12 | h | 8 | h |
| between | | | | | | | | | | | | |
| Applying to Face | | | | | | | | | | | | |
| Carrier of Toner | Paper | | Cotton | | Cotton | | Paper | | Cotton | | Paper | |
| | Mask | | Pad | | Pad | | Mask | | Pad | | Mask | |
| Soaking Time of | 10 | min | 6 | min | 3 | min | 10 | min | 6 | min | 3 | min |
| Carrier | | | | | | | | | | | | |
| Duration of | 30 | min | 20 | min | 10 | min | 30 | min | 20 | min | 10 | min |
| Single Application | | | | | | | | | | | | |
| to Face | | | | | | | | | | | | |
| Temperature for | 35° | C. | 30° | C. | 40° | C. | 35° | C. | 30° | C. | 40° | C. |
| Preheating Toner | | | | | | | | | | | | |
| Isolation layer | Fiber | | Plastic | | Pearlescent | | Fiber | | Plastic | | Pearlescent | |
| | Film | | Film | | Film | | Film | | Film | | Film | |
| Amount of Toner | 5 | ml | 3 | ml | 2 | ml | 5 | ml | 5 | ml | 5 | ml |
| for Use | | | | | | | | | | | | |
| Massage | Pressing | | Gently | | Gently | | Pressing | | Pressing | | Gently | |
| Manipulation | | | Rubbing | | Rubbing | | | | | | Rubbing | |
| Beauty device | Ion | | Microcurrent | | Led Green | | None | | None | | None | |
| | Beauty | | Beauty | | Light | | | | | | | |
| | Importer | | Device | | Beauty | | | | | | | |
| | | | | | Device | | | | | | | |
| Duration of | 20 | min | 5 | min | 15 | min | None | | None | | None | |
| Introducing | | | | | | | | | | | | |
| Treatment | | | | | | | | | | | | |
| Beauty food | Antioxidant | | Qi-blood | | Anti- | | Starch | | Anti- | | Starch | |
| | Pill | | promoting | | sacchari- | | Pill | | sacchari- | | Pill | |
| | | | Pill | | fication | | | | fication | | | |
| | | | | | Pill | | | | Pill | | | |
| Drinking Water | 200 | ml | 300 | ml | 100 | ml | 200 | ml | 200 | ml | 200 | ml |

In order to better facilitate the understanding of the setting of each experimental group by those skilled in the art, Group 1 in Table 1 is taken as an example here. In Group 1, the skin care product was a skin whitening product, the beauty device was an ion beauty importer, and the beauty food was an antioxidant pill. After preheating the toner to 35° C., a mask paper was soaked in 5 mL of the toner for 10 min, and the surface of the mask paper was covered by an isolation layer fiber film. The mask paper was covered on face for 30 min, and the massage manipulation was pressing while applying the paper mask. While performing the external application step, an ion beauty importer was used for 20 min, and the beauty food was taken with 200 mL of drinking water. Wherein, applying the toner on the face once a day, and the time interval between applications of toner was 24 h. The beauty food was taken once a day, and the time interval between oral administrations was 24 h. One of ordinary skill in the art is able to infer the technical solution of other groups according to the detailed description of Group 1, and they will not be repeated herein.

In the present example, the effective ingredient of the skin whitening product comprised one or more of TRICHOLOMA MATSUTAKE extract, CARTHAMUS TINCTORIUS (SAFFLOWER) FLOWER extract, and RHODIOLA ROSEA extract, and the content of the effective ingredient in the skin whitening product was 10-30% by mass; the effective ingredient of the skin brightening product comprised one or more of TRICHOLOMA MATSUTAKE extract, GLYCYRRHIZA URALENSIS extract and PEARL extract, and the content of the effective ingredient in the skin moisturizing product was 10-30% by mass; and the effective ingredient of the skin yellow-removing product comprised one or more of PANAX NOTOGINSENG extract, SALVIA MILTIORRHIZA ROOT extract and ASTRAGALUS MEMBRANACEUS extract, and the content of the effective ingredient in the skin recovering product was 10-30% by mass. The effective ingredient of the antioxidant food comprised one or more of SCHISANDRAE CHINENSIS extract, PANAX QUINQUEFOLIUS extract and BAKUMONDOU EKISU extract, and the content of the effective ingredient in the antioxidant food was 10-30% by mass; the effective ingredient of the qi-blood promoting food comprised one or more of SALVIA MILTIORRHIZA ROOT extract, CRATAEGI FRUCTUS extract and GINKGO BILOBA extract, and the content of the effective ingredient in the collagen-supplementing food was 10-30% by mass; and the effective ingredient of the anti-saccharification food comprised one or more of CANARIUM ALBUM extract, OSMANTHUS FRAGRANS extract and SCUTELLARIAE RADIX extract, and the content of the effective ingredient in the qi-blood promoting food was 10-30% by mass.

The volunteers in each group implemented the corresponding skin care program, and repeated every day. The whole skin care program lasted for 56 days. After successively implementing the skin care program for 56 days, skin condition was recorded on the 56^{th} day. The record of skin condition comprised two parts: professional records, and scores given by a doctor. Comparing the results of the tests on the 56^{th} day with that of the 0^{th} day, the skin condition was recorded according to the proportion of the improvement of effect. The records of the skin condition comprised two parts: professional records, and scores given by a doctor.

Wherein, Table 2 shows the result of professional tests of skin brightness, skin whiteness, melanin content, haematochrome content, lustrousness, mean value of skin color, area of target spot, transparency, area of keratin desquamation, index of keratin desquamation, area of wrinkle, number of pores and water content of the volunteers using professional test tools and test solutions. Table 3 shows the scores subjectively given by the volunteers themselves regarding color of target spot, size of target spot, density of target spot, ruddiness, improvement of skin color, uniformity, whiteness, moistness, lustrousness and brightness, etc., and the full score was 5 points.

TABLE 2

| | Group 1 | Group 2 | Group 3 | Group 4 | Group 5 | Group 6 |
|---|---|---|---|---|---|---|
| L* Value | +4.11% | +3.14% | +3.85% | +1.28% | +0.87% | +0.47% |
| ITA Value | +20.64% | +15.80% | +18.35% | +10.15% | +6.33% | +2.10% |
| Melanin Content | −23.60% | −17.66% | −20.13% | −10.91% | −7.08% | −1.14% |
| Haematochrome | −7.65% | −4.16% | −8.21% | −1.24% | −1.03% | −0.54% |
| Content | | | | | | |
| Lustrousness | +26.38% | +20.55% | +28.42% | +16.62% | +10.23% | +3.54% |
| Mean Value of | −2.58% | −1.60% | −2.33% | −0.99% | −0.84% | −0.07% |
| Skin Color | | | | | | |
| Area of Target | −30.43% | −27.20% | −29.65% | −12.05% | −9.55% | −1.23% |
| Spot | | | | | | |
| Transparency | +4.21% | +3.03% | +4.38% | +1.51% | +1.36% | +0.42% |
| Area of Keratin | −29.89% | −27.14% | −29.19% | −14.71% | −8.55% | −2.11% |
| Desquamation | | | | | | |
| Index of Keratin | −30.87% | −26.48% | −27.76% | −16.05% | −7.91% | −1.06% |
| Desquamation | | | | | | |
| Area of Wrinkle | −17.88% | −21.46% | −20.18% | −9.64% | −5.54% | −2.10% |
| Number of Pores | −10.33% | −12.47% | −11.03% | −5.33% | −3.96% | −1.06% |
| Water Content | +98.11% | +96.54% | +82.62% | +65.40% | +18.24% | +10.39% |

TABLE 3

| | Group 1 | Group 2 | Group 3 | Group 4 | Group 5 | Group 6 |
|---|---|---|---|---|---|---|
| Density of | −3.89% | −2.47% | −3.96% | −1.15% | −1.08% | −0.14% |
| Target Spot | | | | | | |
| Color of | −14.29% | −9.57% | −16.81% | −6.69% | −4.29% | −0.69% |
| Target Spot | | | | | | |
| Size of | −11.40% | −8.81% | −12.58% | −4.07% | −2.13% | −0.27% |
| Target Spot | | | | | | |
| Improvement of | +88.33% | +68.22% | +80.42% | +42.58% | +21.16% | +2.74% |
| Skin Color | | | | | | |
| Ruddiness | +48.88% | +38.89% | +49.61% | +26.33% | +7.23% | +1.07% |
| Uniformity | +27.84% | +24.22% | +25.10% | +15.51% | +7.09% | +0.54% |
| Whiteness | +55.56% | +50.17% | +53.07% | +29.89% | +17.42% | +1.39% |
| Brightness | +93.55% | +85.46% | +87.23% | +60.22% | +15.29% | +2.47% |
| Lustrousness | +88.82% | +76.77% | +74.84% | +42.25% | +22.54% | +3.46% |
| Transparency | +79.09% | +65.55% | +74.92% | +40.13% | +21.39% | +3.09% |
| Moistness | +72.05% | +75.99% | +68.54% | +45.11% | +20.23% | +14.19% |

It can be concluded from Table 2 and Table 3 that comparing with the control group, the skin whitening effect of the test group implementing a technical solution provided by the present disclosure was significantly better than that of the control group from the two aspects of the professional test and individual point of view.

Further, in view that the technical solution provided by the present disclosure had good skin whitening effect, it can be inferred that the technical solution of the present disclosure had effects of facilitating skin recovery. Thus, the technical solution provided by the present disclosure can be widely used in fields of skin care, skin recovery and skin treatment.

In view of above, the present disclosure provides a skin whitening program, comprising: step 1, external application: applying a toner to face 1-3 times each day after cleaning the face, and the time interval between applications is 8-24 h; step 2, introducing treatment: while performing the external application, using an importer to massage the face, after introducing, successively using an essence milk and a cream; step 3, oral administration: after the above steps, orally administering a beauty food for 1-3 times each day, and the time interval between oral administrations is 8-24 h; the skin care product is selected from the group consisting of a skin whitening product, a skin brightening product and a skin yellow-removing product, and a mixture thereof; the beauty device is selected from the group consisting of an ion beauty importer, a microcurrent beauty device, an LED green light beauty device, and a mixture thereof; and the beauty food is selected from the group consisting of an antioxidant food, a qi-blood promoting food and an anti-saccharification food, and a mixture thereof. The present disclosure also provides a use of the above-mentioned skin whitening program in the field of skin care, skin recovery and skin treatment. It can be concluded from the volunteer recruitment test that after treating skin with the program provided by the present disclosure, skin condition of the volunteers improved obviously, and the area of wrinkle on the surface of skin was reduced. The skin whitening program and use thereof provided by the present disclosure solves the technical defects that various skin care methods cannot be reasonably combined to achieve an ideal skin care effect in the conventional art.

The above descriptions are only preferred embodiments of the present disclosure. It should be pointed out that a number of modifications and refinements may be made by one of ordinary skills in the art without departing from the principles of the disclosure, and such modifications and refinements are also considered to be within the scope of protection of the disclosure.

1. A skin whitening program method, comprising
step 1, external application: applying a toner to face 1-3 times each day after cleaning the face, and the time interval between applications is 8-24 h;
step 2, introducing treatment: while performing the external application, using a beauty device to massage the face, then successively using an essence milk and a cream;
step 3, oral administration: after the above steps, orally administering a beauty food for 1-3 times each day, and the time interval between oral administrations is 8-24 h;
the skin care product is selected from the group consisting of a skin whitening product, a skin brightening product and a skin yellow-removing product, and a mixture thereof;
the beauty device is selected from the group consisting of an ion beauty importer, a microcurrent beauty device, an LED green light beauty device, and a mixture thereof; and
the beauty food is selected from the group consisting of an antioxidant food, a qi-blood promoting food, an anti-saccharification food, and a mixture thereof.

2. The skin whitening method according to claim 1, wherein the effective ingredient of the skin whitening product comprises one or more of TRICHOLOMA MATSUTAKE extract, CARTHAMUS TINCTORIUS (SAFFLOWER) FLOWER extract, and RHODIOLA ROSEA extract, and the content of the effective ingredient in the skin whitening product is 10-30% by mass;
the effective ingredient of the skin brightening product comprises one or more of TRICHOLOMA MATSUTAKE extract, GLYCYRRHIZA URALENSIS extract and PEARL extract, and the content of the effective ingredient in the skin brightening product is 10-30% by mass;
the effective ingredient of the skin yellow-removing product comprises one or more of PANAX NOTOGINSENG extract, SALVIA MILTIORRHIZA ROOT extract and ASTRAGALUS MEMBRANACEUS extract, and the content of the effective ingredient in the skin yellow-removing product is 10-30% by mass.

3. The skin whitening method according to claim 1, wherein the effective ingredient of the antioxidant food comprises one or more of SCHISANDRAE CHINENSIS extract, PANAX QUINQUEFOLIUS extract and BAKUMONDOU EKISU extract, and the content of the effective ingredient in the antioxidant food is 10-30% by mass;
the effective ingredient of the qi-blood promoting food comprises one or more of SALVIA MILTIORRHIZA ROOT extract , CRATAEGI FRUCTUS extract and GINKGO BILOBA extract, and the content of the effective ingredient in the qi-blood promoting food is 10-30% by mass; and
the effective ingredient of the anti-saccharification food comprises one or more of CANARIUM ALBUM extract, OSMANTHUS FRAGRANS extract and SCUIELLARIAE RADIX extract, and the content of the effective ingredient in the anti-saccharification promoting food is 10-30% by mass.

4. The skin whitening method according to claim 1, wherein in the external application step, the method of applying toner is: soaking mask paper and/or cotton pad in the toner for 3-10 min, and applying the soaked mask paper and/or cotton pad to the face for 10-30 min.

5. The skin whitening method according to claim 4, wherein in the external application step, an isolation layer is configured on the exterior of the paper mask and/or cotton pad when applying the soaked paper mask and/or cotton pad on the face, and the isolation layer is selected from the group consisting of a plastic film, a fiber film and a pearlescent film.

6. The skin whitening method according to claim 1, wherein in the external application, preheating the toner before applying to the face;
the temperature for preheating is 30-40° C.

7. The skin whitening method according to claim 1, wherein in the external application step, massaging the face by pressing and/or gently rubbing while applying to the face.

8. The skin whitening method according to claim 1, wherein the duration of single introducing treatment is 3-20 min.

9. The skin whitening method according to claim 1, wherein the amount of the toner for single use is 2-5 mL, and in the oral administration step, orally taking the beauty food with 100-300 mL water.

10. (canceled)
